(19) United States
(12) Patent Application Publication
(10) Pub. No.: US 2022/0105192 A1
(43) Pub. Date: Apr. 7, 2022

(54) COMPOSITION, METHOD AND USE THEREOF

(71) Applicant: RYBOQUIN COMPANY LTD, Borders (GB)

(72) Inventors: Simon Newman, Borders (GB); Stephen Hart, Borders (GB); Aristides TAGALAKIS, Borders (GB); Jinhong Meng, Borders (GB)

(21) Appl. No.: 17/423,412

(22) PCT Filed: Jan. 15, 2020
(86) PCT No.: PCT/GB2020/050080

§ 371 Date: Jul. 15, 2021

(30) Foreign Application Priority Data
Jan. 15, 2019 (GB) 1900569.3

(51) Int. Cl. A61K 47/64; C12N 15/113; A61K 47/69; A61K 9/127; A61K 45/06; A61K 31/713; A61P 35/00

(52) U.S. Cl. CPC A61K 47/6455; C12N 15/1137; A61K 47/6911; A61K 9/1271; C12N 2320/32; A61K 45/06; A61K 31/713; A61P 35/00; C12N 2310/14; A61K 9/1272

(57) ABSTRACT

A nanoparticle complex for delivery to cells comprising an RNA interference agent and a delivery vehicle comprised of a liposome and peptide. The peptide may be transmembrane, at least part of which is located external to the liposome. The delivery vehicle is used to deliver the RNA interference agent to cells. The RNA interference agent may target the activity of E3 ubiquitin ligase Itch. The complex may be used to treat chemotherapy resistant tumours and/or cancers. Also related compositions, combination, kit, methods and uses thereof.

### FIELD OF INVENTION

The present invention relates to a composition, a combination or kit, a method of use, and the use thereof. In particular, the present invention relates to compositions, combinations or kits for use in treating cancer, and methods of treating cancer using such compositions, combinations or kits, where RNA interference agents are delivered to a cell.

### BACKGROUND

RNA interference (or RNAi) is a biological process in which RNA molecules inhibit gene expression or translation, by neutralizing targeted mRNA molecules. For RNA interference agents to work, they must reach the mRNA target intact. However, this can be difficult to achieve, particularly in vivo. Whilst various delivery systems have been used in an attempt to deliver RNA interference agents to cells in vitro and/or in vivo, each target and each RNA interference agent presents its own challenges.

RNA interference agents have been suggested as a possible way of targeting drug-resistant tumour cells. For example, p53 mutations are responsible for drug-resistance of tumour cells, which impact the efficacy of treatment, and therefore alternative tumour suppressor pathways have been explored. The E3 ubiquitin ligase ITCH negatively regulates the tumour suppressor protein p73 (TP73), providing a therapeutic target to enhance the sensitivity of the tumour cells to the treatment. This pathway could be targeted using RNA interference agents, provided they can be successfully delivered to the cell. Being able to do so would potentially provide a treatment for otherwise drug-resistant tumour cells, such as those evidenced in pancreatic cancer and neuroblastoma. RNA interference agents, specifically siRNA, for modulating Itch ubiquitinase activity are described in WO2006/077407, which is incorporated herein by reference.

Neuroblastoma is a childhood malignant tumour accounting for ˜13% of all paediatric cancer mortality (Louis and Shohet, 2015). Some types of neuroblastoma are prone to developing drug-resistance during the course of treatment (Lai et al., 2012). One of the genes responsible for this phenomenon is p53 (Xue et al., 2007; Keshelava et al., 2001; Huang et al., 2010; Tweddle et al., 2001), a well-characterized tumour suppressor protein, which binds to various transcriptional factors mediating apoptosis of tumour cells (Vogelstein et al., 2000; Vousden and Lu, 2002). Mutations of p53 lead to compromised tumour cell death and eventually the irresponsiveness to the chemotherapy treatment (Vogelstein et al., 2000; Vousden and Lu, 2002; Xue et al., 2007).

Attempts to restore or reactivate p53 function for tumour therapy (He et al., 2015 ; Bykov and Wiman, 2003; Wiman, 2007; Chen et al., 2014; Ventura et al., 2007; Martins et al., 2006) are under investigation but it may be possible to utilise alternative pathways to compensate for the missing p53 function (Ramadan et al., 2005; Rossi et al., 2004; Venkatanarayan et al., 2016). TP73 is a homologous molecule of p53 and shares significant sequence similarity particularly in the DNA binding domain (DBD), activation domain (AD) and tetramerization domain (TD) (Willis et al., 2003). TP73 shows tumour suppressive activities through its ability to bind transcriptional target genes involved in apoptosis. Overexpression of TP73 promotes the apoptosis of transformed cells. In addition, p73 mutations are infrequent in human cancers (Venkatanarayan et al., 2016) including neuroblastomas (Ejeskar et al., 1999; Ichimiya et al., 1999), making it an attractive gene to manipulate for therapeutic intervention of the p53 null tumours. TP73 is expressed at low level in normal tissues, but may be upregulated in some types of tumours (Yokomizo et al., 1999; Guan et al., 2003; Dominguez et al., 2001; Kovalev et al., 1998) or under conditions where p53 is inactivated (Tophkhane et al., 2012). The expression level of p73 protein is regulated by the E3 ubiquitin ligase ITCH (Rossi et al., 2005) via its ubiquitination pathway, thus, inhibition of ITCH could elevate p73 expression and enhance the chemo-sensitivity of the tumour cells, especially those with defective p53 (Hansen et al., 2007). In addition to p73, Itch also regulates other tumour suppressor genes such as large tumour suppressor 1 (LATS1) (Yeung et al., 2013; Salah et al., 2011), p63 (Rossi et al., 2006; Salah et al., 2013), and RASSFS/NORE1 (Suryaraja et al., 2013).

However, as noted above, delivering RNA interference agents to cells can be challenging, particularly in vivo. Therefore, whilst it is recognised that targeting the ubiquitination pathway of E3 ubiquitin ligase ITCH to modulate the expression of p73 may be a viable way to treat or sensitise tumour cells, this will only be viable if the RNA interference agent can actually reach its target intact. Enabling such delivery would potentially provide a treatment for otherwise drug-resistant tumour cells, such as those evidenced in pancreatic cancer and neuroblastoma.

Therefore, an object of the invention is to provide a composition having an RNA interference agent and a delivery vehicle for delivering the RNA interference agent (such as those described in WO2006/077407) to cells or the like, particularly in vivo. A further object of the invention is to provide treatments for tumours and/or cancers, and for chemotherapy resistant tumours and/or cancers by, for example, modulating the activity of E3 ubiquitin ligase ITCH. A still further object of the invention is to mitigate or obviate issues identified in the field of the invention and with reference to the prior art. Further objects of the invention will be apparent from reading this document.

### DISCLOSURE OF INVENTION

According to a first aspect of the invention, there is provided a composition comprising:
an RNA interference agent; and
a delivery vehicle for delivering the RNA interference agent to a cell, wherein the delivery vehicle comprises:
   a liposome; and
   a peptide.

The delivery vehicle may substantially encapsulate the RNA interference agent.

The liposome may substantially encapsulate the RNA interference agent.

The liposome may encapsulate at least part of the peptide.

The peptide may be a transmembrane peptide, at least part of which is located external to the liposome.

Up to 20 amino acids of the peptide may be external to the liposome, optionally up to 14 amino acids may be external to the liposome, optionally from 7 to 14 amino acids may be external to the liposome.

The composition may be a nanoparticle.

The nanoparticle may have an average diameter of up to approximately 200 nm, optionally up to approximately 150 nm.

The nanoparticle may have an average diameter of from approximately 40 nm to approximately 200 nm, optionally from approximately 40 nm to approximately 150 nm, optionally from approximately 40 nm to approximately 120 nm, optionally from approximately 40 nm to approximately 100 nm, optionally 80 nm to approximately 200 nm, optionally from approximately 80 nm to approximately 150 nm, optionally from approximately 80 nm to approximately 120 nm, optionally from approximately 80 nm to approximately 100 nm.

The nanoparticle may have an average diameter of from approximately 80 nm to approximately 120 nm, optionally approximately 100 nm.

The RNA interference agent may be selected from one or more of the group consisting of: siRNA, miRNA, saRNA and shRNA.

The RNA interference agent may be siRNA.

The RNA interference agent may be configured to modulate the activity of Itch.

The RNA interference agent may be configured to inhibit the activity of Itch.

The RNA interference agent may be configured to down-regulate the expression of ITCH mRNA. The down-regulation of the expression of ITCH mRNA may reduce the level of Itch protein in a cell, thereby inhibiting the activity of Itch protein. Reducing the level of Itch protein in a cell may comprise reducing the amount and/or concentration of Itch protein in a cell.

Itch may be ubiquitin ligase Itch, optionally E3 ubiquitin ligase Itch.

The RNA interference agent may be selected from one or more of the following sequences: 5′-GCUGUUGUUUGCCAUAGAA55-3′ (SEQ ID NO 003 and 5′-UUCUAUGGCAAACAACAGC55-3′ (SEQ ID NO 004.

The liposome may comprise at least one lipid.

The liposome may comprise at least one cationic lipid.

The liposome may comprise at least one phospholipid.

The liposome may comprise at least one cationic lipid and at least one phospholipid.

The at least one phospholipid may comprise a PEG moiety.

The composition may comprise from about 1% by weight to about 5% by weight pegylated phospholipid.

The PEG moiety may have a molecular weight of from about 100 to about 10,000, optionally the PEG moiety has a molecular weight of from about 250 to about 7,500, optionally the PEG moiety has a molecular weight of from about 500 to about 5,000, optionally the PEG moiety has a molecular weight of from about 750 to about 4,000, optionally the PEG moiety has a molecular weight of from about 1,000 to about 3,000, optionally the PEG moiety has a molecular weight of approximately 2,000.

The liposome may comprise at least one cationic lipid, at least one non-pegylated phospholipid and at least one pegylated phospholipid, wherein the ratio of cationic lipid:non-pegylated phospholipid:pegylated phospholipid is approximately 9.5:9.5:1.

The liposome may comprise one or more of the compounds selected from the group consisting of: DOTMA, DOPE, DPPE-PEG, and DPPE-PEG2000.

The peptide may be an integrin targeting peptide.

The peptide may comprise at least one of the following sequences:

(SEQ ID NO 001)
K₁₆GACYGLPHKFCG
and
(SEQ ID NO 002)
K₁₆RVRRGACRGDCLG.

The ratio of lipid:peptide:RNA interference agent may be approximately 1:4:1.

According to a second aspect of the invention, there is provided a composition for modulating the activity of Itch, the composition comprising:
an RNA interference agent configured to modulate the activity of Itch; and
a delivery vehicle for delivering the RNA interference agent to a cell, wherein the delivery vehicle comprises:
   a liposome; and
   a peptide.

The modulation of Itch activity may be inhibition of Itch activity.

The composition of the second aspect may be as described in the first aspect and may include any combination of the variants and embodiments thereof.

According to a third aspect of the invention, there is provided a composition for modulating apoptosis in a cell, the composition comprising:
an RNA interference agent configured to modulate apoptosis in a cell; and
a delivery vehicle for delivering the RNA interference agent to a cell, wherein the delivery vehicle comprises:
   a liposome; and
   a peptide.

The composition of the third aspect may be as described in the first aspect and may include any combination of the variants and embodiments thereof.

According to a fourth aspect of the invention, there is provided a composition for inducing apoptosis in a cell, the composition comprising:
an RNA interference agent configured to induce apoptosis in a cell by modulating the activity of Itch; and
a delivery vehicle for delivering the RNA interference agent to a cell, wherein the delivery vehicle comprises:
   a liposome; and
   a peptide.

The modulation of Itch activity may be inhibition of Itch activity.

The composition of the fourth aspect may be as described in the first aspect and may include any combination of the variants and embodiments thereof.

According to a fifth aspect of the invention there is provided a composition for sensitising cells to cell death, the composition comprising:
an RNA interference agent configured to modulate the activity of Itch; and
a delivery vehicle for delivering the RNA interference agent to a cell, wherein the delivery vehicle comprises:
   a liposome; and
   a peptide.

The modulation of Itch activity may be inhibition of Itch activity.

The composition may further comprise cytotoxic agents configured to induce cell death.

The composition may further comprise DNA damaging agents configured to induce cell death.

The composition of the fifth aspect may be as described in the first aspect and may include any combination of the variants and embodiments thereof.

According to a sixth aspect of the invention there is provided a composition for modulating p63 or p73 stability in a cell, the composition comprising:
an RNA interference agent configured to modulate the activity of Itch; and
a delivery vehicle for delivering the RNA interference agent to a cell, wherein the delivery vehicle comprises:
   a liposome; and
   a peptide.

The modulation of Itch activity may be inhibition of Itch activity.

The composition of the sixth aspect may be as described in the first aspect and may include any combination of the variants and embodiments thereof.

According to a seventh aspect of the invention there is provided a composition comprising:
an RNA interference agent; and
a delivery vehicle for delivering the RNA interference agent to a cell, wherein the delivery vehicle comprises:
   a liposome; and
   a peptide;
      for use as a medicament and/or in therapy.

The composition of the seventh aspect may be the composition of any one of the first, second, third, fourth, fifth and/or sixth aspects and may include any combination of the variants and embodiments thereof.

According to an eighth aspect of the invention there is provided a composition comprising:
an RNA interference agent; and
a delivery vehicle for delivering the RNA interference agent to a cell, wherein the delivery vehicle comprises:
   a liposome; and
   a peptide;
      for use in the treatment of cancer.

The composition of the eighth aspect may be the composition of any one of the first, second, third, fourth, fifth and/or sixth aspects and may include any combination of the variants and embodiments thereof.

According to a ninth aspect of the invention there is provided a combination or kit comprising:
a composition comprising:
   an RNA interference agent; and
   a delivery vehicle for delivering the RNA interference agent to a cell, wherein the delivery vehicle comprises:
      a liposome, and
      a peptide; and
a cytotoxic agent configured to induce cell death.

The combination or kit may comprise DNA damaging agents configured to induce cell death.

The composition of the combination or kit of the ninth aspect may be the composition of any one of the first, second, third, fourth, fifth and/or sixth aspects and may include any combination of the variants and embodiments thereof.

According to a tenth aspect of the invention there is provided a combination or a kit comprising:
a composition comprising:
   an RNA interference agent; and
   a delivery vehicle for delivering the RNA interference agent to a cell, wherein the delivery vehicle comprises:
      a liposome, and
      a peptide; and
a cytotoxic agent configured to induce cell death;
   for use as a medicament and/or in therapy.

The combination or kit may comprise DNA damaging agents configured to induce cell death.

The composition of the combination or kit of the tenth aspect may be the composition of any one of the first, second, third, fourth, fifth and/or sixth aspects and may include any combination of the variants and embodiments thereof.

According to an eleventh aspect of the invention there is provided a combination or a kit comprising:
a composition comprising:
   an RNA interference agent; and
   a delivery vehicle for delivering the RNA interference agent to a cell, wherein the delivery vehicle comprises:
      a liposome, and
      a peptide; and
a cytotoxic agent configured to induce cell death;
   for use in the treatment of cancer.

The composition of the combination or kit of the eleventh aspect may be the composition of any one of the first, second, third, fourth, fifth and/or sixth aspects and may include any combination of the variants and embodiments thereof.

According to a twelfth aspect of the invention there is provided a method of treating cancer, the method comprising the step of administering to a patient a therapeutically effective amount of the composition of any one of the first, second, third, fourth, fifth, sixth, seventh and/or eighth aspects and/or any combination of the variants and embodiments thereof; or the combination or kit of any one of the ninth, tenth and/or eleventh aspects and/or any combination of the variants and embodiments thereof.

The method may comprise at least one other modality of treatment of cancer.

The at least one other modality of treatment of cancer may be radiotherapy.

According to a thirteenth aspect of the invention, there is provided a method of apoptosing cells, the method comprising the step of using the composition of the first, second, third, fourth, fifth, sixth, seventh and/or eighth aspects and/or any combination of the variants and embodiments thereof; or the combination or kit of any one of the ninth, tenth and/or eleventh aspects and/or any combination of the variants and embodiments thereof.

According to a fourteenth aspect of the invention, there is provided a method of modulating p63 or p73 stability in a cell, the method comprising the step of using the composition of the first, second, third, fourth, fifth, sixth, seventh and/or eighth aspects and/or any combination of the variants and embodiments thereof; or the combination or kit of any one of the ninth, tenth and/or eleventh aspects and/or any combination of the variants and embodiments thereof.

According to a fifteenth aspect of the invention, there is provided a method of increasing the sensitivity of a tumour cell to a chemotherapeutic agent comprising the step of using the composition of the first, second, third, fourth, fifth, sixth, seventh and/or eighth aspects and/or any combination of the variants and embodiments thereof; or the combination or kit of any one of the ninth, tenth and/or eleventh aspects and/or any combination of the variants and embodiments thereof.

According to a sixteenth aspect of the invention, there is provided the use of the composition of the first, second, third, fourth, fifth, sixth, seventh and/or eighth aspects and/or any combination of the variants and embodiments thereof; or the combination or kit of any one of the ninth, tenth and/or eleventh aspects and/or any combination of the variants and embodiments thereof.

According to a seventeenth aspect of the invention, there is provided the use of the composition of the first, second, third, fourth, fifth, sixth, seventh and/or eighth aspects and/or any combination of the variants and embodiments thereof; or the combination or kit of any one of the ninth, tenth and/or eleventh aspects and/or any combination of the variants and embodiments thereof, as a medicament and/or in therapy.

According to an eighteenth aspect of the invention there is provided the use of the composition of the first, second, third, fourth, fifth, sixth, seventh and/or eighth aspects and/or any combination of the variants and embodiments thereof; or the combination or kit of any one of the ninth, tenth and/or eleventh aspects and/or any combination of the variants and embodiments thereof, in the treatment of cancer.

The use may comprise at least one other modality of treatment of cancer.

The at least one other modality of treatment of cancer may be radiotherapy.

According to a nineteenth aspect of the invention, there is provided the use of the composition of the first, second, third, fourth, fifth, sixth, seventh and/or eighth aspects and/or any combination of the variants and embodiments thereof; or the combination or kit of any one of the ninth, tenth and/or eleventh aspects and/or any combination of the variants and embodiments thereof, in the apoptosing of cells.

According to a twentieth aspect of the invention, there is provided the use of the composition of the first, second, third, fourth, fifth, sixth, seventh and/or eighth aspects and/or any combination of the variants and embodiments thereof; or the combination or kit of any one of the ninth, tenth and/or eleventh aspects and/or any combination of the variants and embodiments thereof, in the modulation of p63 or p73 stability in a cell

According to a twenty-first aspect of the invention, there is provided the use of the composition of the first, second, third, fourth, fifth, sixth, seventh and/or eighth aspects and/or any combination of the variants and embodiments thereof; or the combination or kit of any one of the ninth, tenth and/or eleventh aspects and/or any combination of the variants and embodiments thereof, in increasing the sensitivity of a tumour cell to a chemotherapeutic agent.

By the term “nanoparticle” it is meant a particle having a diameter of from about 10 nm to about 1,000 nm (Saudi Pharm J. 2011 Jul; 19(3): 129141). Particles having a diameter, or range of diameters, within about 10 nm to about 1,000 nm may also be nanoparticles.

A liposome is a vesicle (normally spherical) having at least one lipid bilayer.

It will be understood that the components of the composition are chosen such that the total amount is 100% by weight.

The alternative features and different embodiments as described apply to each and every aspect and each and every embodiment thereof mutatis mutandis.

### BRIEF DESCRIPTION OF THE DRAWINGS

Embodiments of the invention will now be described, by way of example, with reference to the drawings, in which:

FIG. 1 shows expression of ITCH and TP73 in neuroblastoma cell lines. A: RT-PCR and the qPCR results of the expression in Kelly cells and BE2 cells, B: immunostaining showing the expression of Itch and TP73 at the protein level;

FIG. 2 shows expression of integrin αv, β3 and β5 in neuroblastoma cells. a: RT-PCR; b: western blot and c: immunostaining all showed the presence of these integrin molecules in neuroblastoma cell lines Kelly and BE2;

FIG. 3 shows Knockdown of ITCH in vitro using lipofectAMINE2000 (A and B) or nanoparticles (C). A: qPCR of Kelly cells (A-a, b) and BE2 cells (A-c, d) transfected with different concentration of ITCH siRNA (A-a, c) or using different amount of lipofectAMINE 2000 reagent (A-b, d). B: western blot showed the knockdown of ITCH protein by siRNA transfection in Kelly cells (B-a) or BE2 cells (B-b). L2K=lipofectAMINE 2000 only. C: qPCR experiment showing the expression of ITCH mRNA on Kelly cells after transfection of 100 nM ITCH siRNA with different nanoparticle formulations for 4 hours (left panel) or 48 hours (right panel). Cells transfected with LipofectAMINE 2000 (L2K) were used as control.

FIG. 4 shows knockdown of Itch causes the upregulation of TP73 (A) and induces apoptosis of the Kelly cells upon irradiation. A: Western blot showing the silencing of Itch protein and upregulation of TP73 after transfection of ITCH siRNA in Kelly cells. 1, 3, 5, 7 and 9 are samples transfected with ITCH siRNA; 2, 4, 6, 8 and 10 are samples transfected with irrelevant siRNA. D1, D2, D3 and D6 suggested days after transfection, and D6+3 are samples which have had a second transfection 3 days after the 1st transfection. B: Apoptotic cells after irradiation of Kelly cells which have been transfected with ITCH siRNA (b) and irrelevant siRNA (a). Figure c showed the quantification of percentage of SubG1 cells within each treatment group;

FIG. 5 shows ITCH expression in tumours 24 h and 48 h after ITCH siRNA treatment. A: Relative ITCH expression in each treatment group. B: Individual sample responses to the ITCH siRNA treatment at 24 h or 48 h. There were no differences between the treatment group and the control group 24 h after treatment. There were more samples with lower ITCH expression in the treated group than the control group 48 h after treatment;

FIG. 6 shows percentage of tumour samples of each treatment group which express ITCH mRNA relatively lower than 1, 0.9, 0.8 and 0.7, respectively.

### DETAILED DESCRIPTION

Experiments were undertaken to determine whether a delivery vehicle could be used to successfully deliver RNA interference agents to tumour cells using delivery vehicles (in this case a liposome at least partly encapsulating a peptide), and whether this could in turn successfully modulate ITCH expression to enhance the sensitivity of the tumour cells to treatment. To do this, two p53 null neuroblastoma cell lines were used as in vitro models, and siRNA techniques were applied to downregulate ITCH expression. Furthermore, utilizing nanoparticles (de la Fuente et al., 2015;Hart, 2010), the in vivo silencing efficacy of the candidate ITCH siRNAs in a neuroblastoma xenograft model (Valentiner et al., 2008) was tested.

Two p53-mutant neuroblastoma cell lines were used as in vitro models. The expression of ITCH and the in vitro silencing efficacy by ITCH siRNA was tested by immunostaining, western blot and qPCR. Corresponding expression of TP73 and the response of tumour cells to irradiation after ITCH silencing were examined using western blot and flow cytometry. Furthermore, using nanoparticles, in vivo silencing efficacy of the ITCH siRNA on a mouse xenograft model of neuroblastoma was also examined.

### Materials and Methods

Materials: 1,2-di-O-octadecenyl-3-trimethylammonium propane (DOTMA), 1,2-dipalmitoyl-sn-glycero-3-phosphoethanolamine-N-[methoxy(polyethylene glycol)-2000] (DPPE-PEG2000) and 1,2-dioleoyl-sn-glycero-3-phosphoethanolamine (DOPE) were purchased from Avanti Polar Lipids, Inc. (Alabaster, Ala., USA). Peptide Y (K₁₆GACYGLPHKFCG (SEQ ID NO 001)) was synthesized by China Peptides Co., Ltd. (Shanghai, People's Republic of China).

Cell culture: two p53-mutant neuroblastoma cell lines, Kelly (Horvilleur et al., 2008;Gogolin et al., 2013) and SK-N-BE-2 (BE2)(Tweddle et al., 2001) were maintained in vitro in RPMI-1640 medium (Sigma) supplemented with 2 μM Glutamine and 10% FBS (Fetal Bovine Serum). Cells were split every 3-4 days.

Transfection with LipofectAMINE 2000: one day before transfection, cells were plated at 8×10⁴ cells per well for 24-well plate or 1.5×10⁵ cells per well for 6-well plate in culture medium. On the day of transfection, LipofectAMINE 2000 (Thermal Fisher) or ITCH/Irrelevant siRNA (Eurogentec) was pre-diluted in OptiMEM (Thermal Fisher) with various concentrations and mixed to form complexes at room temperature (RT) for 25 min before adding to cells. mRNAs or protein lysates were collected 72 hours after transfection for further analysis.

Transfection with nanoparticles in vitro: the basic formulation of the nanoparticle was Lipid:peptide:siRNA. In this study, there was a comparison of 3 different lipids which contain different concentrations of PEG. 1) DOTMA/DOPE (DD), contains no PEG; 2) AT-1, DD which also contains 1% PEG (molar ratio); 3) GK27, DD which also contains 5% PEG (molar ratio). The peptide used in the study was ME27 (K₁₆RVRRGACRGDCLG (SEQ ID NO 002)). The siRNA used was ITCH siRNA (sense strand: 5′-GCUGUUGUUUGCCAUAGAA55-3′ (SEQ ID NO 003)); antisense strand: 5′-UUCUAUGGCAAACAACAGC55-3′ (SEQ ID NO 004)) (Eurogentec) and Irrelevant siRNA (Dharmacon). The ratio of the Lipid:peptide:siRNA was 1:4:1, and the siRNA concentration was 100 nM for transfection. Kelly cells were prepared at 8×10⁴ cells/well in 24-well plate one day before transfection. Nanoparticles were carefully prepared and mixed in the order of Lipid+Peptide+siRNA, and left at RT for 30 min in order to form complexes before adding to the cells. The whole plate was centrifuged at 1500 rpm for 5 min to allow the nanoparticles to settle and incubated for either 4 hours or 48 hours before analysis.

Semi-quantitative RT-PCR: mRNA of cultured cells was extracted using RNeasy mini kit (Qiagen). mRNA concentration was determined by Nano drop 3.1 software. One-step RT-PCR kit (Qiagen) was used to perform the semi-quantitative RT-PCR, using 10 ng mRNA from each sample. The sequence of the primers were:

ITCH forward:
(SEQ ID NO 005)
5′-ACCGGCTGCCATCTTAGTCT-3′;
ITCH reverse:
(SEQ ID NO 006)
5′-GGAAAACCTGAAGTTCTCACAGT-3′;
beta-actin forward:
(SEQ ID NO 007)
5′-GCCCTGAGGCACTCTTCCA-3′;
beta-actin reverse:
(SEQ ID NO 008)
5′-ATGCCACAGGACTCCATGC-3′;
SDHA forward:
(SEQ ID NO 009)
5′-TGGGAACAAGAGGGCATCTG-3′;
and
SDHA reverse:
(SEQ ID NO 010)
5′-CCACCACTGCATCAAATTCATG-3′.

Real-time PCR: cDNA from each mRNA sample was synthesized using superscript III first strand synthesis kit (Thermal Fisher). To determine the ITCH expression, real time PCR was performed using SYBR-green qPCR kit (Eurogentec), using beta-actin or SDHA (sequence details as above) as endogenous control. The qPCR was run using either StepOne plus qPCR machine (Thermal Fisher) or CFX96 qPCR system (Bio-Rad). Results were analysed using their corresponding software.

Immunostaining on cultured cells: cells were plated on poly-lysine coated coverslips at a density of 5×10⁴ cells/ml. 24 hours later the cells were fixed with 4% PFA (Paraformaldehyde) for 10 min at RT. After one hour blocking with 10% NGS (Normal Goat Serum), the cells were incubated with antibodies against ITCH (BD, 1:100), TP73 (Generon, 1:500), integrin αv (R&D, 1:100), β3 (Generon, 1:100) and β5(R&D, 1:100) at 4° C. overnight, followed by one hour incubation with Alexa-488 or Alexa-594 conjugated corresponding secondary antibodies (1:500, Thermal Fisher). The coverslips were then mounted with hydromount (Sigma) mounting medium containing 10 μg/ml DAPI and images were acquired using Metamorph software.

Western blot: 72 hours after transfection, protein was extracted in RIPA (Radio-Immunoprecipitation Assay) lysis buffer (Sigma) containing a complete protease inhibitor cocktail (1:100, Roche). 100 μl of lysis buffer was added to each well of the 6-well plate, and left on ice for 10 minutes. Samples were collected and boiled for 3 minutes and then centrifuged at 14,000 g for 10 minutes at 4° C. The supernatants were stored at −80° C. until needed. 30 μl/well of each sample were loaded onto NuPAGE Novex 10% bis-Tris Gel, and run at a constant voltage of 150V for 1 hour, before being transferred to a nitrocellulose membrane at 300 mA for 2 hours. The membrane was then blocked with Odyssey block solution (LI-COR Biosciences) for 60 min, incubated by primary antibodies of mouse anti-ITCH antibody (1:2000, BD) or rabbit anti-TP73 (1:1000, Generon), and mouse anti-beta actin (1:5000, Sigma) overnight at 4° C. After washing with PBS (phosphate-buffered saline) containing 1% Tween 20 (PBST) for 15 min×3 times at room temperature, the membrane was then incubated with IRDye 680 RD goat anti-rabbit and IRDye 800 CW goat anti-mouse 2^{nd} antibodies (1:15000, LI-COR Biosciences) for 1 hour at RT. The image of the blotted membrane was acquired by Odyssey Clx infrared imaging system (LI-COR Biosciences) using image studio software.

For western blotting of integrins, the primary antibodies used were: goat anti-human integrin alpha V (1:1000, R&D) or mouse anti-human integrin beta 3 (1:1000, R&D) or Sheep anti-human integrin beta 5 (1:1000, R&D) followed by corresponding anti-goat/sheep/mouse HRP conjugated antibody (1:5000, R&D). The membrane was then incubated with chemiluminescent reagent and visualized.

SubG1 analysis: to examine the effects of ITCH silencing on the apoptosis of neuroblastoma cells upon irradiation, Kelly cells were firstly transfected with ITCH siRNA as described above, using irrelevant siRNA as control. Cells were exposed to 4 Gy irradiation three days after transfection. 24 hours after irradiation, cells were trypsinized and centrifuged at 2000 rpm, for 5 min. Pre-cold 70% ethanol was then added dropwise to cell pellet, and the cells were fixed at 4° C. for 30 min before centrifuge again and the cell pellet washed with citric buffer twice before adding 50 μl RNase followed by 50 μg/ml PI (propidium iodide) for 5 min. The cells were then pelleted again at 2000 rpm for 5 min, and re-suspended in 0.5 ml PBS for FACS (fluorescent associated cell sorting) analysis. The cell cycle profile was acquired using FACS calibur machine and the percentage of cells in the SubG1 region was calculated using Flowjo software.

Establishment of the xenograft neuroblastoma model and in vivo transfection of ITCH siRNA with nanoparticles: 6-8 week old NSG-SCID female mice were ordered from Charles River laboratories. All procedures were approved by UCL animal care policies and were carried out under Home Office Licenses issued in accordance with the United Kingdom Animals (Scientific Procedures) Act 1986 (UK). The mice were acclimatized for 1-2 weeks before engrafting the neuroblastoma cells. On the day of injection, Kelly cells were trypsinized and re-suspended in medium at a density of 3×10⁶ cells/100 μl. 100 μl cell suspension was mixed with equal volume of Matrigel (BD biosciences) on ice and immediately injected subcutaneously into the left or right upper thigh of the mouse. The body weight and the tumour size/volume of each mouse were monitored from D4 after engraftment. The in vivo administration of the ITCH siRNA nanoparticles was performed on mouse once its tumour grew bigger than 5mm at both dimensions. The nanoparticle formulation for in vivo study was also prepared at the 1:4:1 ratio, using cationic lipid AT-1, which contains 1% PEG and the peptide used was ME27. 1 mg siRNA/kg of nanoparticles was injected intravenously into each mouse. The tumour was harvested either 24 hours or 48 hours after administration, and processed for qPCR analysis.

Real time RT-PCR analysis of tumour samples: each tumour was divided into 4 parts and mRNA was extracted respectively for real time RT-PCR analysis. iTaq Universal SYBR Green One-Step Kit was used in this assay. For normalization, 2 separate housekeeping genes, beta-actin and SDHA (Fisher M, 2005) were included. As there were in total 104 samples to test, it was not possible to fit all samples in a single 96 well testing plate. Therefore, sample 05 c was chosen, from an untreated tumour sample as a common control. This sample was included in every plate that was run and all the data from each plate were calculated relative to this sample (the ddCT value of each sample was calculated by subtracting the deICT value of sample 05 c).

### Results

1. Expression of ITCH and TP73 in Neuroblastoma Cell Lines:

To determine the optimal in vitro cell culture model for this project, two p53 mutant neuroblastoma cell lines, Kelly and BE2 cells, were chosen and semi-quantitative RT-PCR, real time RT-PCR and immunostaining were performed to determine the expression levels of ITCH and TP73 in these two cell lines.

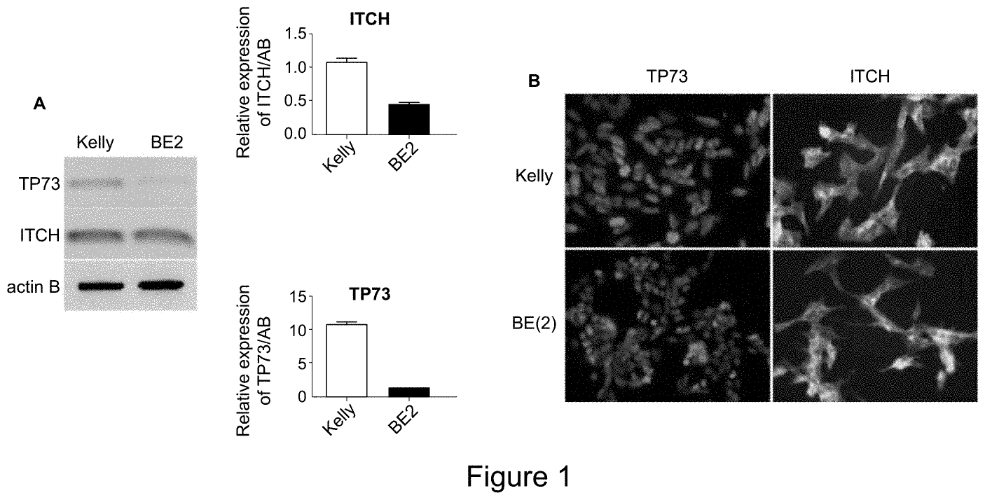

As shown in FIG. 1, RT-PCR on mRNA extracted from the proliferating Kelly cells and BE2 cells showed that both these two cell lines expressed ITCH and TP73. Real-time PCR suggested that Kelly cells express higher level of ITCH and TP73 than BE2 cells (FIG. 1A). Immunostaining showed that both cell lines also expressed Itch and TP73 protein (FIG. 1B). Therefore, both cell lines could be used for transfections with ITCH siRNA in order to knockdown ITCH expression.

### 2. Expression of Integrin αv, β3 and β5 on Neuroblastoma Cells:

It has been shown that nanoparticles containing peptide, which contains an integrin-targeting RGD motif, can be an effective delivery tool for in vivo tumour targeting (Grosse et al., 2010) and it was intended to use the same formulation for the in vivo silencing experiment. Thus, it was important to establish that the tumour cells expressed integrin receptor proteins to enable the specific targeting of the tumour by nanoparticles. Therefore, the expression of the specific ME27 ligands, integrins αv, β3 and β5 in neuroblastoma cells was examined by RT-PCR, immunostaining and western blot analysis.

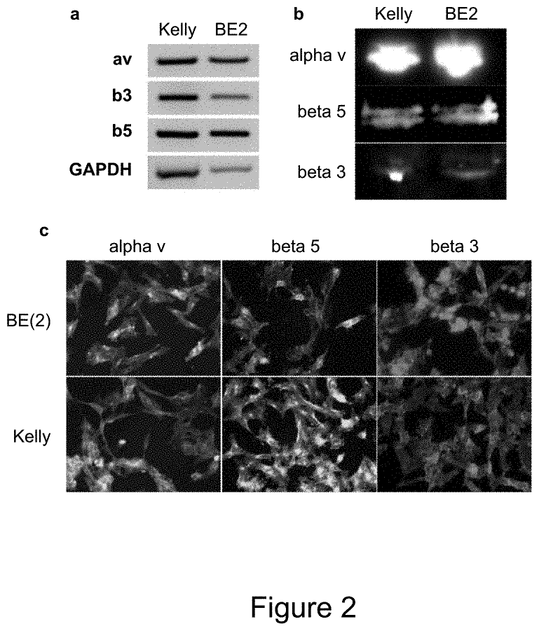

As shown in FIG. 2, it was found that both Kelly and BE2 cells expressed integrins αv, β3 and β5 at the mRNA level (RT-PCR, FIG. 2A) and protein level (immunostaining, western blot, FIG. 2c, 2b). This result suggested that these neuroblastoma cells can be targeted by the nanoparticles via the interaction between the ME27 peptide and integrins.

### 3. In Vitro Silencing of ITCH in Neuroblastoma Cell Lines:

### 3.1. Transfection Using LipofectAMINE 2000 Reagent:

To determine ITCH silencing in neuroblastoma cells in vitro, neuroblastoma cells were transfected with ITCH siRNA, using LipofectAMINE 2000 as the transfection reagent. Cells were transfected with either, a) ITCH siRNA (6.25 nM to 100 nM) using 2 μl/well LipofectAMINE 2000 or b) Different amounts of LipofectAMINE2000 (0.5 μl/well, 1 μl/well and 2 μl/well) with 100 nM ITCH siRNA for 4 hours in OptiMEM, and then change into complete medium for a further 72 hours.

The ITCH mRNA levels were determined by qPCR, and cells transfected with irrelevant siRNA were used as the control.

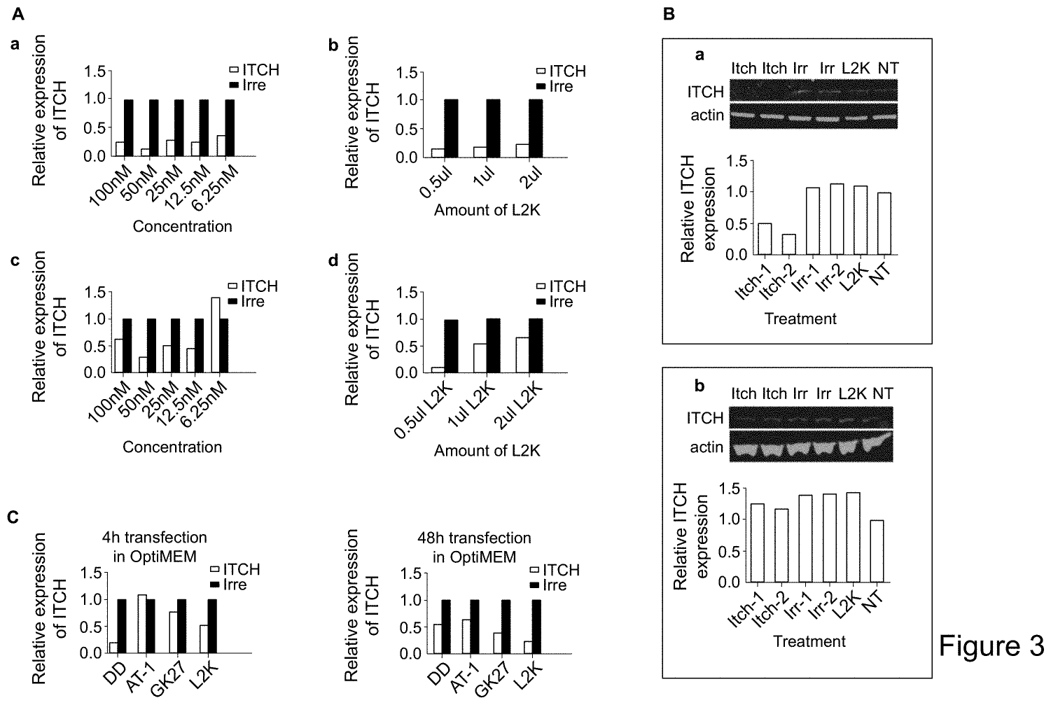

It was found that:
a) In Kelly cells, after transfection, there were 78%, 89%, 73.5%, 77.8% and 64.7% ITCH knockdown in 100 nM, 50 nM, 25 nM, 12.5 nM and 6.25 nM ITCH SiRNA transfected groups, respectively. In BE2 cells, there were 38.2%, 71.8%, 48.7%, 54% and -39% ITCH knockdown in 100 nM, 50 nM, 25 nM, 12.5 nM and 6.25 nM ITCH SiRNA transfected groups, respectively. ITCH expression in both Kelly cells and BE2 cells was effectively knocked down using low concentrations (as low as 12.5 nM tested) of ITCH siRNA, as shown in FIG. 3A-a, c. This is important as lower concentrations would enable the avoidance of unnecessary toxicity caused by the siRNA. There was no significant dose-dependent effect when the concentration of ITCH siRNA increased.
b) There was no difference in ITCH knockdown using different amount of LipofectAMINE 2000 for transfection (FIG. 3A-b, d). In Kelly cells, using 100nM SiRNA, after transfection, there were 85%, 83% and 78.8% ITCH knockdown in cells transfected with 0.5 μl/well, 1 μl/well and 2 μl/well LipofectAMINE 2000, respectively. In BE2 cells, there were 88.7%, 49% and 34% ITCH knockdown in cells transfected with 0.5 μl/well, 1 μl/well and 2 μl/well LipofectAMINE 2000, respectively. Again, this result provides evidence that the lower amount of transfection reagent (0.5 ul/well) could achieve similar transfection efficiency levels to higher amount of reagent (2 ul/well), which would diminish possible toxicity to the cells.

Next the extent of ITCH silencing at the protein level in Kelly cells and BE2 cells was tested. As shown in FIG. 3B, 72 hours after transfection, the remaining Itch protein level was 31.6% in ITCH siRNA transfected Kelly cells (FIG. 3B-a), in comparison to non-transfected cells. However, there were no changes in Itch protein level in treated BE2 cells in comparison to irrelevant siRNA treated cells (FIG. 3B-b).

Based on the western blot results from the Kelly and BE2 cells, it was determined that the Kelly cell line was more responsive to the ITCH siRNA treatment than the BE2 cell line, in terms of silencing at the protein level. So, in the in vivo experiment that followed, Kelly cells were chosen to establish the xenograft models.

### 3.2. Transfection Using Nanoparticles:

Kelly cells were transfected with nanoparticles containing lipids formulated with different concentration of polyethylene glycol (PEG) (Grosse et al., 2010). Three types of lipids were used as following: DOTMA/DOPE, with no PEG; AT-1, containing 1% PEG and GK27, which contains 5% PEG. Kelly cells were plated in 24-well plates at a density of 8×10⁴ cells/well. On the day of the transfection, cells were transfected with the following formulations: 1) DOTMA/DOPE: ME27:ITCH siRNA; 2) AT-1:ME27:ITCH siRNA and 3) GK27:ME27:ITCH siRNA, all at a ratio of 1:4:1. Cells transfected with irrelevant siRNA with corresponding formulations were used as negative controls. Cells transfected with LipofectAMINE 2000 and ITCH siRNA were also included as a positive control. The concentration of siRNA used in this experiment was 100nM. All transfections were performed in OptiMEM and 2 time points were compared in this experiment: 4 hours (nanocomplexes left for 4 hours on cells and then replaced with complete media) and 48 hours after transfection (nanocomplexes left for 48 hours on cells in complete media). As shown in FIG. 3C, when the cells were transfected for 4 hours, there was 70% knockdown of ITCH in the DOTMA/DOPE:ME27:ITCH siRNA transfected group. However, AT-1:ME27:ITCH siRNA and GK27:ME27:ITCH siRNA transfected groups achieved either none or small amount of (22% knockdown) ITCH silencing. However, when the cells were transfected for 48 hours, which means that the nanoparticles stay in the medium throughout the experiment, there was around 44% knockdown of ITCH in DOTMA/DOPE:ME27:ITCH siRNA transfected group, 37% knockdown in AT-1:ME27:ITCH siRNA transfected group, and around 61% knockdown of ITCH in GK27:ME27:ITCH siRNA transfected group. These data suggested that for DOTMA/DOPE:ME27:ITCH siRNA formulation, extending the incubation time did not enhance the transfection efficiency; while for AT-1:ME27:ITCH siRNA and GK27:ME27:ITCH siRNA formulations, increasing the transfection time significantly enhanced transfection efficiency.

### 4. Knockdown of ITCH Upregulates TP73 in Kelly Cells

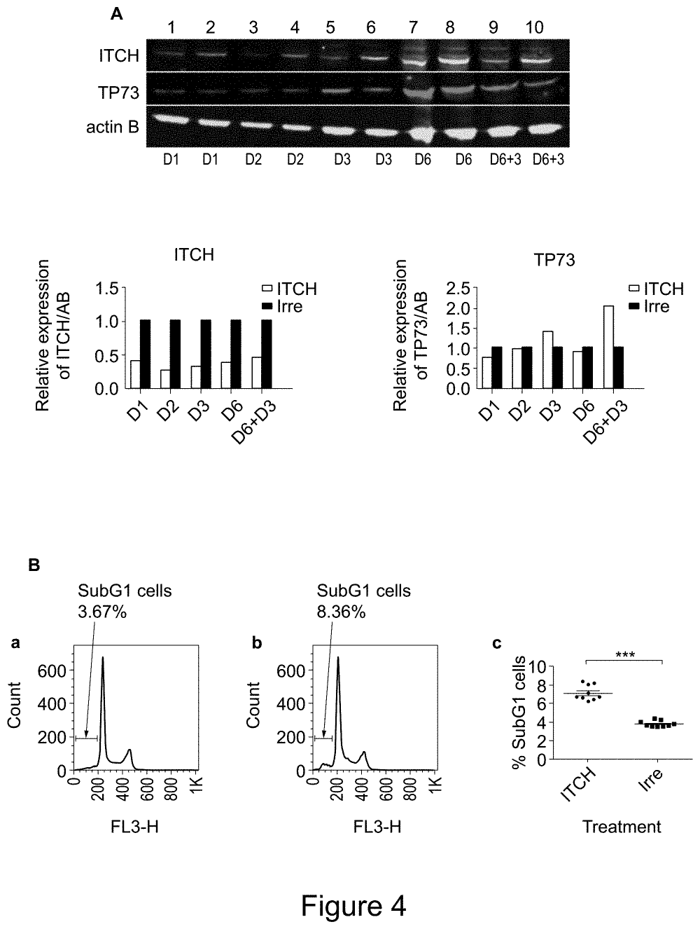

Kelly cells were transfected with ITCH siRNA or irrelevant siRNA in 6-well plates using LipofectAMINE2000. The protein samples were collected 1 day, 2 days, 3 days and 6 days after transfection. In some wells, Kelly cells were subjected to a 2nd transfection 3 days after the first transfection, and samples were collected at 6 days after the first transfection, i.e., 3 days after the 2nd transfection, these samples were thus labelled as D6+3. Western blot analysis with the Itch and TP73 antibodies was performed using the above samples, and the expression of each protein was normalized to β-actin (FIG. 4).

As expected, downregulation of Itch protein was observed in samples which had been transfected with ITCH siRNA, in comparison to samples which were transfected with irrelevant siRNA, from 1 day to 6 days after the 1st transfection. However, TP73 upregulation was only detected at day 3 samples and day 6 samples which had been transfected twice

(D6+3). These data suggest that the TP73 upregulation happens after the Itch downregulation, and it is important to keep the Itch protein at lower level (2 transfections) in order to maintain the elevated TP73, as suggested by D6+3 samples.

### 5. Knockdown of ITCH Sensitizes Kelly Cells to Irradiation

Kelly cells were transfected with ITCH and irrelevant siRNA, respectively. Three days later, cells were subjected to 4 Gy irradiation. The subG1 population was analysed one day after the irradiation. As shown in FIG. 4B, one day after irradiation, cells which have been transfected with ITCH siRNA contained 8.36% apoptotic cells, while cells which have been transfected with irrelevant siRNA contained 3.56% SubG1 cells. This difference was statistically significant between the 2 groups (p<0.001, Mann-Whitney test).

This shows that silencing of ITCH by siRNA significantly induces apoptosis of Kelly neuroblastoma cells upon irradiation. It is thought that upon silencing of ITCH, the tumour suppressor protein TP73 is stabilized, which theoretically for p53 mutant cells, triggers the apoptotic pathway upon the irradiation treatment. The treatment could also be drug treatment (chemotherapy).

### 6. In Vivo Silencing of ITCH in Neuroblastoma Xenografts

### 6.1. The Relative Expression of ITCH in the Xenograft Tumours was Decreased at 48 h After Transfection, not at 24 h

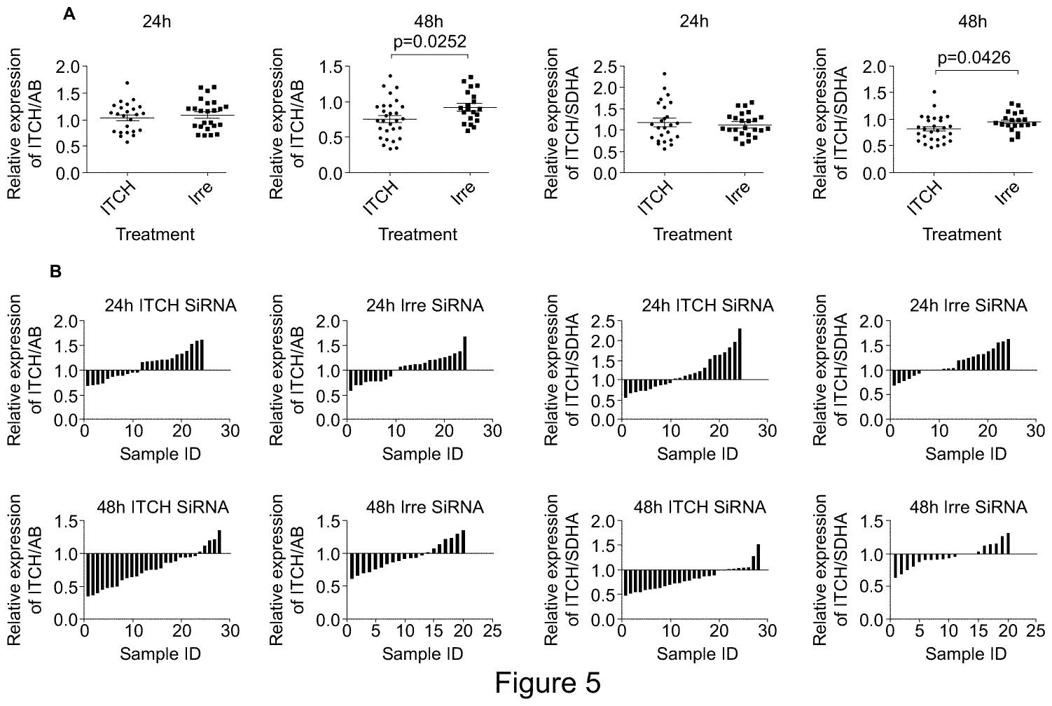

As shown in table 1, the relative expression of ITCH at 24 h in the ITCH siRNA treated group was similar to that in the irrelevant siRNA treated group, when the data were normalized to either β-actin or SDHA (Fischer et al., 2005), there were no statistical differences between these 2 groups (FIG. 5A) by either normalization method. However, 48 hours after delivery, it was found that the relative expression of ITCH was significantly lower in the treated group than in the control group (FIG. 5A), when the data were normalized to β-actin (p=0.0252, 19.15% silencing) or SDHA (p=0.0426, 14.6% silencing).

TABLE 1
The relative expression of ITCH in the xenograft tumours

| | Relative expression of | | Relative expression of | |
|---|---|---|---|---|
| | ITCH/β-actin | | ITCH/SDHA | |
| | (mean ± SEM) | | (mean ± SEM) | |
| | 24 hours | 48 hours | 24 hours | 48 hours |
| ITCH SiRNA | 1.05 ± 0.06 | 0.76 ± 0.05 | 1.18 ± 0.10 | 0.82 ± 0.05 |
| Irrelevant | 1.10 ± 0.06 | 0.94 ± 0.05 | 1.13 ± 0.06 | 0.96 ± 0.04 |
| SiRNA | | | | |
| P value | p > 0.05 | p = 0.0252 | p > 0.05 | p = 0.0426 |

6.2. Individual Sample Response to ITCH SiRNA Treatment Showed That More Samples in the Treated Group 48 h After Treatment Express Lower Levels of ITCH mRNA

All the individual samples we plotted for their expression of ITCH. FIG. 5B shows that at 24 hours later there were no differences in the number of samples which expressed lower levels of ITCH between the ITCH siRNA treated group and irrelevant siRNA treated group. However, there were significantly more samples in the treated group 48 h after treatment that showed decreased ITCH expression than in the control group. The tendency is the same when the samples were normalized with β-actin or SDHA (FIG. 5B).

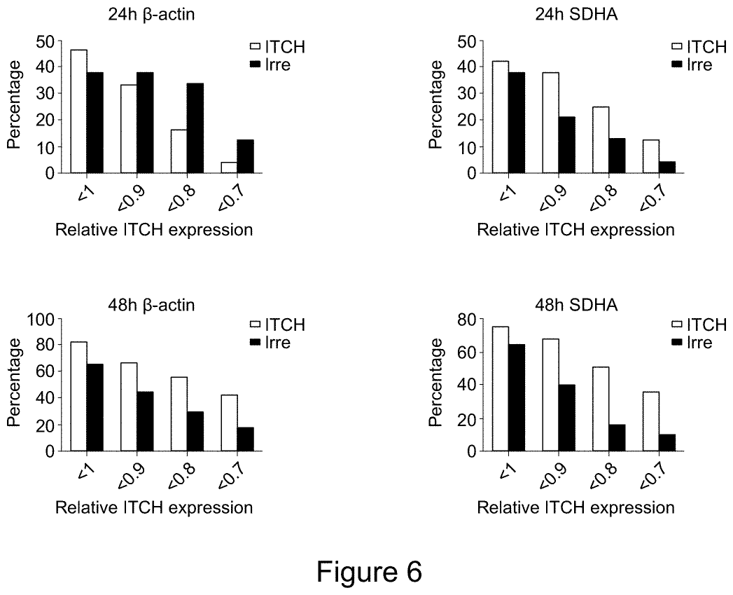

The percentage of low-ITCH expression samples in each group was compared. The percentage of low-ITCH expression samples (relative expression lower than 1 or 0.8) within each group was calculated, and the differences between treated and control group was shown in table 2, 3 and FIG. 6. It was found that 24 hours after delivery, there were only slightly more or even less samples expressing lower level of ITCH in treated group, while 48 h after delivery, there were 27.14% or 35% more samples expressing lower level of ITCH (relative expression <0.8) in treated group than in control group, when the data was normalized to β-actin or SDHA, respectively.

TABLE 2
ITCH expression 24 h after injection

| Group | | ITCH SiRNA | | Irre SiRNA | | Differ- |
|---|---|---|---|---|---|---|
| n number | | 24 | | 24 | | ences |
| Relative ITCH expression | | Number | % | Number | % | in % |
| Normalized | <1 | 11 | 45.83 | 9 | 37.5 | 8.33 |
| to β-actin | <0.8 | 4 | 16.67 | 8 | 33.33 | −16.67 |
| Normalized | <1 | 10 | 41.67 | 9 | 37.5 | 4.17 |
| to SDHA | <0.8 | 6 | 25 | 3 | 12.5 | 12.5 |

### DISCUSSION

Mutation of the tumour suppressor gene p53 is the major reason for the recurrence of drug-resistant neuroblastoma, and some other cancers too, occurring in over half of all solid tumours. In fact, TP53 mutations occur in almost every type of cancer at rates from 38%-50% in ovarian, oesophageal, colorectal, head and neck, larynx, and lung cancers to about 5% in primary leukaemia, sarcoma, testicular cancer, malignant melanoma, and cervical cancer (FIG. 1). Mutations are more frequent in advanced stage or in cancer subtypes with aggressive behaviour (such as triple negative or HER2-amplified breast cancers) (Wang et al. 2004a; Wang et al. 2004b; Langerod et al. 2007). In cancers with low mutation rates, p53 is often inactivated by alternative mechanisms. This is the case for cervical cancer in which p53 is targeted for degradation by HPV E6 (Tommasino et al. 2003) or for sarcoma that overexpress amplified HDM2 (see Cold Spring Harb Perspect Biol. 2010 Jan; 2(1) for more information).

In addition to restoring the functional p53 pathway, manipulation of alternative tumour suppressor pathways may provide therapeutic benefit for treatment of these types of tumours. It is thought that elevating the TP73 protein, a homologous protein of p53 superfamily, by inhibiting its ubiquitous pathway, may elicit the apoptosis of p53 mutant neuroblastoma cells. Therefore, the compositions of the present invention were designed to target Itch, the E3 ubiquitous ligase responsible for the degradation of a panel of tumour suppressor genes including TP73.

As illustrated in the experiments herein, Itch and TP73 are expressed in two p53 mutant neuroblastoma cell lines, with Kelly cells expressing higher levels of both Itch and TP73 than BE2 cells (FIG. 1), suggesting the existence of a compensating tumour suppressor pathway in the absence of functional p53 in these cell lines, and the feasibility of manipulating this pathway to induce the programmed cell death of tumour cells. Itch, an E3 ubiquitin ligase, is the key enzyme responsible for ubiquitin and degrading the TP73 protein (Rossi et al., 2005).

Downregulation of Itch expression in tumour cells thus provides therapeutic value for treatment of the p53 mutant tumours. In the above in vitro transfection experiment, although both cell types could be transfected and achieve silencing of ITCH at the mRNA level, BE2 cells failed to respond at the protein level (FIG. 3B). Therefore, the focus was on Kelly cells as the in vitro and in vivo model in the following studies. The TP73 gene has two distinct promoters coding for two protein isoforms with opposite effects: the transactivation proficient TAp73 shows pro-apoptotic effects and the amino-terminal-deleted DeltaNp73 has an anti-apoptotic function. The cellular outcome of the tumour cells upon treatment is finely regulated by the balance between TAp73 and DeltaNp73 in the p53 mutant cells (Ramadan et al., 2005). It has been shown that the major isoform of TP73 expressed in Kelly cells was the TAp73 (Horvilleur et al., 2008), whose upregulation would enhance the apoptosis of the cells upon treatment. The western blot using a p73 antibody also confirmed the single TAp73 band at molecular weight of around 73 KD (FIG. 4A). After ITCH silencing, the TP73 was upregulated at the protein level in Kelly cells and consequently these cells which were initially unresponsive, underwent apoptosis upon irradiation, as indicated by an increased SubG1 population (FIG. 4B). However, it is noticeable that the upregulation of TP73 protein was not as dramatic as the extent of the ITCH silencing. This is not surprising as TP73 is not the sole protein regulated by Itch. Other molecules such as TP63 (Rossi et al., 2006), large tumour suppressor 1 (LATS1) (Yeung et al., 2013; Ho et al., 2011; Salah et al., 2011), and RASSFS/NORE1 (Suryaraja et al., 2013) were also reported to be negatively regulated by Itch. Despite the fact that TP73 was not dramatically upregulated upon ITCH silencing, increased apoptosis in ITCH siRNA transfected Kelly cells was detected compared to irrelevant siRNA treated cells, showing that this strategy is a potential therapeutic treatment.

In the above experiments, receptor-targeted liposome-peptide-siRNA compositions in the form of nanoparticles were used to deliver the ITCH siRNA in vivo. The formulation of the compositions contained peptide ME27, which can mediate the specific targeting via its interaction with integrin αv, β3 and β5 on tumour cells. It was found that these integrins were present in neuroblastoma cell lines (FIG. 2), suggesting that compositions formulated with the design used could be effectively delivered to tumour sites via the receptor-peptide interaction after in vivo delivery into neuroblastoma xenograft model.

In addition to determining the expression of integrins in neuroblastoma cell lines, in vitro transfections with nanoparticles were performed where the transfection efficiency of the lipid formulations which contained 1% PEG (AT-1), 5% PEG (GK27) and no PEG (DOTMA/DOPE) were compared. The presence of PEG has been shown to stabilize the compositions/nanoparticles in serum in vivo, preventing aggregation or disassembly (Tagalakis et al., 2014a). It was found that the lipid formulations containing PEG (AT-1 and GK27) did not work as well as the lipid without PEG (DOTMA/DOPE) in 4 hour transfections (FIG. 3C), although at longer incubation times (48 hour), ITCH siRNA silencing was evident, suggesting a slow releasing process with these nanoparticles in cell culture.

The in vivo results showed a similar tendency (slow release of siRNA from the nanoparticle complexes). 24 hours after transfection, there was no silencing detected in treated groups, while 48 hours after transfection silencing was detectable by qPCR, with statistically lower levels of ITCH expression in the ITCH siRNA treated group than in the irrelevant siRNA treated group (FIG. 5A). There were also more samples expressing lower ITCH mRNA in the treated group than in the control group 48 hours after transfection (FIG. 5B). This result was consistent with the in vitro findings showing the therapeutic effect of manipulating ITCH expression in the treatment of drug-resistance neuroblastoma and other drug-resistant cancers.

### SUMMARY

The compositions and method of the present invention enabled delivery of siRNA to cells, resulting in the ITCH mRNA being expressed in p53-mutant neuroblastoma cell lines being silenced by siRNA transfection. The TP73 protein was stabilized 3 days after ITCH siRNA transfection, which mediated the apoptosis of the neuroblastoma cells upon irradiation. Delivery of the ITCH siRNA using compositions of the present invention in the form of nanoparticles to the neuroblastoma xenograft model showed around 15-20% ITCH silencing 48 hours after transfection.

Using the compositions and methods of the present invention, ITCH can be silenced both in vitro and in vivo in p53 mutant neuroblastoma cells. Silencing of ITCH sensitizes the tumour cells to treatment, and chemotherapy/radiotherapy treatment could therefore be used in combination with this silencing. Thus, the compositions and methods of the present invention can be used to enhance therapeutic effects on neuroblastoma and other chemotherapy resistant tumours.

For example, the results of the experiments carried out using the compositions and methods described herein show that ITCH can be effectively silenced in neuroblastoma both in vitro and in vivo. Silencing of ITCH in vitro stabilizes TP73 protein on neuroblastoma cells and sensitized the cells to irradiation treatment. The results show that this strategy is feasible for combining with the conventional chemo-/radio-therapy to treat the drug-resistant p53-null neuroblastomas and other chemotherapy resistant tumours.

Therefore, the use of the composition and method as described herein provides a viable way of delivering RNA interference agents to tumour and/or cancerous cells, and a method of treating otherwise drug-resistant cancers.

Various modifications and variations to the described embodiments of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes of carrying out the invention which are obvious to those skilled in the art are intended to be covered by the present invention.

SEQ ID NOs.
SEQ ID NO 001:
K₁₆GACYGLPHKFCG
Sequence Listing Free Text: Peptide Y
SEQ ID NO 002:
K₁₆RVRRGACRGDCLG
Sequence Listing Free Text: ME27
SEQ ID NO 003:
5′-GCUGUUGUUUGCCAUAGAA55-3′
Sequence Listing Free Text: ITCH siRNA sense
strand
SEQ ID NO 004:
5′-UUCUAUGGCAAACAACAGC55-3′
Sequence Listing Free Text: ITCH siRNA antisense
strand
SEQ ID NO 005:
5′-ACCGGCTGCCATCTTAGTCT-3′
Sequence Listing Free Text: ITCH forward sequence
(RT-PCR)
SEQ ID NO 006:
5′-GGAAAACCTGAAGTTCTCACAGT-3′
Sequence Listing Free Text: ITCH reverse sequence
(RT-PCR)
SEQ ID NO 007:
5′-GCCCTGAGGCACTCTTCCA-3′
Sequence Listing Free Text: beta-actin forward
sequence (RT-PCR)
SEQ ID NO 008:
5′-ATGCCACAGGACTCCATGC-3′
Sequence Listing Free Text: beta-actin reverse
sequence (RT-PCR)
SEQ ID NO 009:
5′-TGGGAACAAGAGGGCATCTG-3′
Sequence Listing Free Text: SDHA forward sequence
(RT-PCR)
SEQ ID NO 010:
5′-CCACCACTGCATCAAATTCATG-3′
Sequence Listing Free Text: SDHA reverse sequence
(RT-PCR)

1. A composition comprising:
an RNA interference agent; and
a delivery vehicle for delivering the RNA interference agent to a cell, wherein the delivery vehicle comprises:
a liposome; and
a peptide.

2. The composition of claim 1, wherein the delivery vehicle substantially encapsulates the RNA interference agent.

3. The composition of claim 1, wherein the liposome substantially encapsulates the RNA interference agent.

4. The composition of claim 1, wherein the liposome encapsulates at least part of the peptide.

5. The composition of claim 1, wherein the peptide is a transmembrane peptide, at least part of which is located external to the liposome.

6. The composition of claim 5, wherein up to 20 amino acids of the peptide are external to the liposome.

7. The composition of claim 1, wherein the composition is a nanoparticle.

8-9. (canceled)

10. The composition of claim 7, wherein the nanoparticle has an average diameter of from approximately 80 nm to approximately 120 nm.

11. The composition of claim 1, wherein the RNA interference agent is selected from one or more of the group consisting of: siRNA, miRNA, saRNA and shRNA.

12. (canceled)

13. A composition comprising:
an RNA interference agent; and
a delivery vehicle for delivering the RNA interference agent to a cell, wherein the delivery vehicle comprises:
a liposome; and
a peptide, wherein the RNA interference agent is configured to modulate the activity of Itch.

14. The composition of claim 13, wherein the RNA interference agent is configured to inhibit the activity of Itch.

15. The composition of claim 13, wherein Itch is ubiquitin ligase Itch.

16. The composition of claim 15, wherein Itch is E3 ubiquitin ligase Itch.

17. The composition of claim 1, wherein the RNA interference agent is selected from one or more of the following sequences: 5′-GCUGUUGUUUGCCAUAGAA55-3′ (SEQ ID NO 003) and 5′-UUCUAUGGCAAACAACAGC55-3′ (SEQ ID NO 004).

18-24. (canceled)

25. The composition of claim 1, wherein the liposome comprises at least one cationic lipid, at least one non-pegylated phospholipid and at least one pegylated phospholipid, wherein the ratio of cationic lipid:non-pegylated phospholipid:pegylated phospholipid is approximately 9.5:9.5:1

26. The composition of claim 1, wherein the liposome comprises one or more of the compounds selected from the group consisting of: DOTMA, DOPE, DPPE-PEG, and DPPE-PEG2000.

27. The composition of claim 1, wherein the peptide is an integrin targeting peptide.

28. The composition of claim 1, wherein the peptide comprises at least one of the following sequences: [K16]GACYGLPHKFCG (SEQ ID NO 001) and [K16]RVRRGACRGDCLG (SEQ ID NO 002).

29-32. (canceled)

33. The composition of claim 1,
wherein the RNA interference agent is configured to modulate apoptosis in a cell.

34. (canceled)

35. The composition of claim 1,
an wherein the RNA interference agent is configured to induce apoptosis in a cell by modulating the activity of Itch.

36. The composition of claim 35, wherein the modulation of Itch activity is inhibition of Itch activity.

37-39. (canceled)

40. The composition of claim 35, wherein the composition further comprises cytotoxic agents configured to induce cell death.

41. The composition of claim 35, wherein the composition further comprises DNA damaging agents configured to induce cell death.

42-49. (canceled)

50. A combination or kit comprising:
a composition comprising:
an RNA interference agent; and
a delivery vehicle for delivering the RNA interference agent to a cell, wherein the delivery vehicle comprises:
a liposome, and
a peptide; and
a cytotoxic agent configured to induce cell death.

51. The combination or kit of claim 50, wherein the combination or kit comprises DNA damaging agents configured to induce cell death.

52-58. (canceled)

59. A method of treating cancer, the method comprising the step of administering to a patient a therapeutically effective amount of the composition of claim 1.

60. The method of claim 59, wherein the method comprises at least one other modality of treatment of cancer.

61. The method of claim 60, wherein the at least one other modality of treatment of cancer is radiotherapy.

62. A method of apoptosing cells, the method comprising the step of administering to cells to be apoptosed the composition of claim 1.

63. A method of modulating p63 or p73 stability in a cell, the method comprising the step of administering to the cell the composition of claim 1.

64. A method of increasing the sensitivity of a tumour cell to a chemotherapeutic agent comprising the step of administering to a tumour cell the composition of claim 1.

65-72. (canceled)


SEQUENCE LISTING (10 sequences)

SEQ ID NO: 1 - protein, 28 aa, Artificial Sequence
  Source - Peptide y
KKKKKKKKKKKKKKKKGACYGLPHKFCG

SEQ ID NO: 2 - protein, 29 aa, Artificial Sequence
  ME27
KKKKKKKKKKKKKKKKRVRRGACRGDCLG

SEQ ID NO: 3 - RNA, 19 nt, Artificial Sequence
  ITCH siRNA sense strand
gcuguuguuugccauagaa

SEQ ID NO: 4 - RNA, 19 nt, Artificial Sequence
  ITCH siRNA antisense strand
uucuauggcaaacaacagc

SEQ ID NO: 5 - DNA, 20 bp, Artificial Sequence
  ITCH forward sequence (RT-PCR)
accggctgccatcttagtct

SEQ ID NO: 6 - DNA, 23 bp, Artificial Sequence
  ITCH reverse sequence (RT-PCR)
ggaaaacctgaagttctcacagt

SEQ ID NO: 7 - DNA, 19 bp, Artificial Sequence
  beta-actin forward sequence (RT-PCR)
gccctgaggcactcttcca

SEQ ID NO: 8 - DNA, 19 bp, Artificial Sequence
  beta-actin reverse sequence (RT-PCR)
atgccacaggactccatgc

SEQ ID NO: 9 - DNA, 20 bp, Artificial Sequence
  SDHA forward sequence (RT-PCR)
tgggaacaagagggcatctg

SEQ ID NO: 10 - DNA, 22 bp, Artificial Sequence
  SDHA reverse sequence (RT-PCR)
ccaccactgcatcaaattcatg